(19) United States
(12) Patent Application Publication
(10) Pub. No.: US 2026/0248454 A1
(43) Pub. Date: Aug. 27, 2026

(54) IN-LINE FLUID MONITOR

(71) Applicant: University of Central Oklahoma, Edmond, OK (US)

(72) Inventor: Maurice HAFF, Edmond, OK (US)

(21) Appl. No.: 19/059,588

(22) Filed: Feb. 21, 2025

(51) Int. Cl. A61B 5/00; A61B 5/145; A61M 25/00; A61M 27/00; G01N 21/77; G01N 21/78; G01N 33/52

(52) U.S. Cl. CPC A61B 5/6852; A61B 5/14507; A61B 5/742; A61B 5/746; A61M 25/0017; A61M 27/00; G01N 21/78; G01N 33/528; A61B 2562/0285; A61B 2562/12; A61B 2562/162; G01N 2021/7763

(57) ABSTRACT

The present invention provides a method of manufacture for an in-line fluid monitor, and its use in monitoring fluid extractions for pathogen invasion, and indicating pathogen presence in urinary catheters and surgical drains at critical thresholds. The in-line fluid monitor comprises a capsule encapsulating a nanofiber biosensor that exhibits a color-change response to the presence of at least one pathogen produced biomarker. The capsule may be transparent and can be connected to a catheter via a first conduit and connected via a second conduit to a fluid collector. A fluid passing through the first conduit into the capsule and out of the capsule though the second conduit exposes the biosensor in the capsule to the fluid passing through the capsule, and any color-change response to pathogen presence is observable.

### FIELD OF INVENTION

The present disclosure relates generally to chromatic biosensors for use in diagnostics and identification of pathogens in fluids. More specifically, the present disclosure relates to monitoring fluids during catheter use in medical applications.

All of the references, patents. and patent applications that are referred to herein are incorporated by reference in their entirety as if they had each been set forth herein in full. Note that this application is one in a series of applications by the Applicant covering methods and apparatus for enabling biomedical applications of nanofibers. The term “fiber” and the term “nanofiber” may be used interchangeably, and neither term is limiting. The disclosure herein goes beyond that needed to support the claims of the particular invention set forth herein. This is not to be construed that the inventor is thereby releasing the unclaimed disclosure and subject matter into the public domain. Rather, it is intended that patent applications will be filed to cover all of the subject matter disclosed below. Also, please note that the terms frequently used below “the invention” or “this invention” is not meant to be construed that there is only one invention being discussed. Instead, when the terms “the invention” or “this invention” are used, it is referring to the particular invention being discussed in the paragraph where the term is used.

### BACKGROUND

The use of catheters is common for a range of medical use cases. These uses include, but are not limited to, indwelling and intermittent urinary catheterization, as well as surgical site drainage. Both urinary catheters and surgical drainage catheters are associated with possible complications, one of the most frequently occurring of which is catheter-associated infection. In addition, the longer a catheter remains in the body, the more likely bacterial infection will result, while intermittent urinary catheterization has a significant risk of introducing pathogens into the body that result in infection.

Urinary tract infections (UTIs) are a severe public health problem and are caused by a range of pathogens, but most commonly by Escherichia coli, Klebsiella pneumoniae, Proteus mirabilis, Enterococcus faecalis and Staphylococcus saprophyticus.

Urinary tract infections (UTIs) are a common medical complaint. It is estimated that up to 40 percent of women will have a UTI at some time in their lives. Also called bladder infections or cystitis, a UTI occurs when bacteria enter the bladder, usually through the urethra (urine tube), and begin to multiply.

Urine contains fluids, salts and waste products but is sterile or free of bacteria, viruses and other disease-causing organisms. A UTI occurs when bacteria from another source, such as the nearby anus, gets into the urethra. The most common bacteria found to cause UTIs is Escherichia coli (E. coli). Other bacteria can cause UTI, but E. coli is the culprit about 90 percent of the time.

All urinary tract infections (UTIs) are among the most common presenting causes of sepsis in hospitals and long-term care (LTC) facilities. Some are simple UTIs that can be managed with outpatient antibiotics and lead to almost universally good outcomes. However, complicated urinary tract infections may lead to florid urosepsis, which can be fatal. Several risk factors can complicate urinary tract infections and lead to treatment failure, repeat infections, or significant morbidity and mortality. It is vitally important to determine if the patient's infection may have resulted from one of these risk factors and whether the episode is likely to resolve with first-line antibiotics. Complicated urinary tract infections are those that present with greater morbidity, carry a higher risk of treatment failure, and typically require longer antibiotic courses, frequently requiring additional workup. Complicated urinary tract infections include those that occur: in males, in pregnant females (including asymptomatic bacteriuria), as a result of obstruction, hydronephrosis, renal tract calculi, or colovesical fistula, in immunocompromised patients or the elderly, due to atypical organisms, after instrumentation, involve urinary catheters, in renal transplant patients, in patients with impaired renal function, after prostatectomies or radiotherapy. Additionally, urinary tract infections that recur despite adequate treatment are considered complicated. Early detection of bacterial presence at low thresholds enables early intervention to prevent worsening of infection.

Proteus mirabilis, a Gram-negative rod-shaped bacterium most noted for its swarming motility and urease activity, frequently causes catheter-associated urinary tract infections (CAUTIs) that are often polymicrobial. These infections may be accompanied by urolithiasis, the development of bladder or kidney stones due to alkalinization of urine from urease-catalyzed urea hydrolysis. During infection, histological damage is caused by cytotoxins including hemolysin and a variety of proteases, some autotransported.

It has been estimated that ~100 million indwelling urinary catheters (IUC) are sold annually worldwide. In the US alone, ~30 million urinary catheters are fitted each year, making IUCs by far the most commonly deployed medical device, with levels of use far outstripping other common devices such as central venous catheters or fracture fixation devices. Although in many cases the use of IUCs can benefit patients and greatly aid treatment and recovery, these devices undermine the innate barriers to bacterial colonization naturally present in the urinary tract, thus predisposing patients to infection by uropathogenic bacteria. Given the widespread use of these devices, catheter associated urinary tract infections (CAUTIs) are currently among the most common nosocomial infections in many healthcare settings. CAUTIs pose a serious risk to patient welfare and a significant financial burden to health service providers, with estimated costs of up to $424-451 million per annum in the USA.

The problem of CAUTI is particularly pronounced in patients who are managed long-term with urethral catheterization, where IUC are in place for weeks or months at a time. This includes many elderly individuals and those with spinal cord injuries, in whom urethral catheterization is often used to manage incontinence in a community care setting. One of the most problematic and severe complications arising from CAUTI in this group is the encrustation and blockage of catheters, which may be experienced by up to 50% of patients undergoing long-term urethral catheterization. Encrustation and blockage is almost exclusively due to infection by Proteus mirabilis, which is isolated from up to 45% of CAUTIs.

Catheter blockage causes painful retention of urine within the bladder, and subsequent vesico-ureteric reflux of infected urine to the kidneys. If blockage is not detected before this occurs, patients suffer episodes of severe kidney infection and septicaemia. Unfortunately, as the majority of long-term catheterized patients are cared for in the community, where constant clinical surveillance is not available, blockage typically remains unnoticed until life threatening consequences arise, and hospital treatment is required. Although a range of catheters impregnated with antimicrobials are widely available, their use in controlling infection even during short-term catheterization (<7 days) remains questionable

The concept of using urinary pH elevation to provide infection responsive drug release has been explored and successfully achieved controlled release of nalidixic acid from poly(2-hydroxyethylmethacrylate (p(HEMA)) hydrogels. Approaches have been described for an “early warning” system designed to alert patients and carers of forthcoming catheter blockage. These systems take the form of an infection-responsive surface coating, compatible with existing catheter designs, able to provide a visual warning of P. mirabilis infection prior to encrustation and blockage. These coatings consist of a dual-layered polymeric architecture, in which a lower layer of hydrogel (poly (vinyl-alcohol)) is employed to encapsulate the self-quenching dye 5(6)-carboxyfluorescein, at concentrations sufficient to inhibit fluorescence. This lower layer is capped and sealed by an upper pH-sensitive ‘trigger’ layer, ensuring no dye release while this is in place. The ‘trigger’ layer is composed of EUDRAGIT® S 100 (an anionic co-polymer of methacrylic acid and methyl methacrylate). Elevation of urinary pH upon P. mirabilis infection (via the urease-catalysed hydrolysis of urea) causes dissolution of the upper EUDRAGIT® S 100 layer, releasing the carboxyfluorescein contained in the lower hydrogel matrix to provide a clear visual signal throughout the catheter drainage system that blockage is imminent, and intervention is required. Disadvantages of surface coating a catheter system include at least limited single pathogen (P. mirabilis) detection, response dependent on pH level which may vary widely due to causes other than pathogens, and application required during device manufacturing preventing use in standard catheter devices.

Intermittent catheterization (IC) is the preferred procedure for individuals with incomplete bladder emptying from non-neurogenic or neurogenic lower urinary tract dysfunction (NLUTD). IC is now considered the gold standard for bladder emptying in individuals following spinal cord injury (SCI) who have sufficient manual dexterity. Despite improvements in catheter material and properties, the most frequent complication of IC is a catheter-associated urinary tract infection (CAUTI), where an 84% overall prevalence of self-reported complications are associated with IC in wheelchair athletes. Of these wheelchair athletes with a SCI, median duration of performing IC was 10 years. Twenty-seven percent sustained urethral injuries and 63% had at least one episode of UTI during the last 12 months. More than 40% of these patients reused their catheters which exposed them to an increased risk for UTIs. Ninety percent of all urethral injuries were reported by individuals with a cervical SCI. Almost one-fourth of male athletes had a history of inflammation/infection of genital organs associated with IC.

UTI is the most frequent complication in patients performing IC. Catheter-associated UTI (CAUTIs) is of concern because when urethral damage occurs, the mucosal barrier to infection is compromised. The bladder wall is susceptible to bacteria that circulate in retained urine. When the bladder becomes stretched from retained urine, the capillaries become occluded, preventing the delivery of metabolic and immune substrates to the bladder wall. Systematic reviews indicate that the frequency of people experiencing 1 or more UTIs per year while undertaking IC in the community is substantial, ranging from 15.4% to 86.6% per year.

Many persons with SCI who use urinary catheters for bladder management have bacterial colonization of their bladder. These patients are at higher risk than the general population for developing a UTI and renal deterioration. UTIs can be the result of re-use of the same catheter for multiple catheterizations, poor catheterization technique, or the passing of the catheter through a normally very contaminated area of the urethra before the catheter reaches the bladder. It has been demonstrated that a single catheter insertion can initiate a CAUTI.

A CAUTI may also be caused by the formation of biofilms (micro-organisms that colonize the internal surface of catheters). In patients with indwelling urinary catheters, these biofilms can contain up to 16 different strains of bacteria! Under unfavorable conditions, such as re-use of a catheter, organisms can detach from the biofilm and become free floating in the urine, which can lead to symptomatic infection. Reused catheters become contaminated by debris and by microorganisms, some with biofilm. In a large (N=912) community-based population of individuals with SCI, half of which were women, women reported a significantly greater number of UTIs than men (p=0.003). Predictor factors of UTI included high mean catheterization volumes and non-self-catheterization (someone other than the patient performs the catheterization).

Surgical drainage is a frequent use case for catheters. The placement of a drainage catheter in the surgical site is common in surgical procedures. Although a surgical drainage catheter is expected to reduce postoperative collections and identify postoperative complications, it facilitates the entry of bacteria into the clean wound area. Thus, surgical drainage catheters are associated with possible complications, such as surgical drain infections (SDIs). In addition, the longer the drain remains in the body, the more likely bacteria are to be isolated from the drainage fluid. Surgical site infections (SSIs) occur frequently and impact patients and health care systems. Remote surveillance of surgical wounds is currently limited by the need for manual assessment by clinicians.

A patient may require drain placement for various reasons. Often, they are placed at the end of a surgery or percutaneously to help eliminate any fluid that may accumulate within the wound. A common type of surgical drain is the Jackson-Pratt®. Certain organs may require a drain to assist with the removal of their contents, such as foley catheters or nasogastric tubes. Drains may also be placed to help remove fluid or air from body cavities. A chest tube is a good example of this type of drain. If a patient develops an abscess, a drain is often required to help remove the infected fluid more quickly. Excess fluid in the surgical site can cause significant pain as well as injury to surrounding tissues and organs. Excess fluid can also increase the risk of infection.

Drains are often described as being active or passive. Passive drains allow use gravity to help remove excess fluid, without needing additional pressure. An example of a passive drain would be placing a foley catheter to gravity or using a penrose drain. A penrose drain is a relatively flat, ribbon-like tube that creates a passage from a wound to the open air, which allows any excess fluid to simply flow outward. The area surrounding the opening is often lightly covered with gauze to collect fluid as it drains and must be changed when saturated.

Active drains use actual pressure, typically negative pressure, to help remove excess fluid from the body. Examples of an active drainage system would be a Jackson-Pratt (JP)® drain or Hemovac®. With both types of drains, and the plurality of other negative pressure wound drainage systems available, the pressure is created by compressing the collection container, which creates a low-pressure vacuum that pulls the fluid out of the body.

Drains can also be described as open or closed. An open system simply means that it is open to air. An example of an open system would be a penrose drain, as described above. A closed drain, on the other hand, is not open to the environment. Rather, the draining fluid is contained within the system, and the collection bulb or bag is simply emptied from time to time, as needed. Each of these systems utilize a tube to transport fluid from a wound to a collection point.

Evaluation and management of complicated infections in the urinary tract and in surgical sites is an essential role of interprofessional team members in collaborating to provide well-coordinated care and enhance outcomes for affected patients in all care settings. The sensitive, selective, and rapid detection of clinical pathogens is a critical step in the prevention/control of pathogenic outbreaks, and timely treatment of bacterial and fungal infections, Conventional bacterial diagnostic approaches such as bacterial culture, morphologie analysis, biochemical staining, enzyme-linked immunosorbent assay, and polymerase chain reaction are time consuming and entail complex pretreatment procedures, preparation/enrichment of samples, advanced analytical equipment, and skillful technicians. These conventional diagnostic approaches present significant challenges for achieving a timely treatment response in any care setting, but is particularly challenging in resource-limited environments including long-term care facilities and at-home settings. Observation of clinical signs and symptoms is often fallible, particularly for the unpracticed care provider and patient. There is, therefore, an urgent need to develop new sensors and methods widely usable in all catheter and fluid extraction systems that address the current challenges in detection and discrimination of clinically relevant concentrations of bacteria and fungi, with high specificity and sensitivity, in a short period of time (e.g., a few minutes to a few hours).

### SUMMARY

Pathogenic bacteria and fungi secrete various enzymes that help them invade host tissues and evade the immune system. Some key enzymes include the following:
Pathogenic Bacteria:
   1. Proteases: Break down proteins in host tissues, aiding in invasion and nutrient acquisition.
   2. Lipases: Degrade lipids, helping bacteria to penetrate fatty tissues.
   3. Hyaluronidase: Breaks down hyaluronic acid in connective tissues, facilitating the spread of bacteria.
   4. Collagenase: Degrades collagen, a major component of connective tissues, aiding in tissue invasion.
   5. Coagulase: Causes blood clotting, which can protect bacteria from immune cells.
Pathogenic Fungi:
   1. Pectinases: Degrade pectin, a component of plant cell walls, facilitating tissue penetration.
   2. Proteases: Similar to bacterial proteases, these break down proteins in host tissues.
   3. Lipases: Help fungi to degrade lipids and invade fatty tissues.
   4. Chitinases: Break down chitin, a component of fungal cell walls, which can help in remodeling fungal structures during infection.

These enzymes play crucial roles in the pathogenicity of bacteria and fungi, enabling them to infect and cause disease in their host. Dyes responsive to these enzymes can be used to indicate though a color-change the presence of pathogens in a wound or on human skin. Organic dyes responsive to pathogen produced enzymes, such as proteases and lipases, are designed to undergo a visible color change upon interaction with these enzymes.

Fluorogenic dyes emit fluorescence upon cleavage by specific enzymes. For instance, MCA (7-methoxycoumarin-4-acetic acid) and EDANS (5-((2-aminoethyl)amino)naphthalene-1-sulfonic acid) are used in fluorogenic assays to study inflammatory caspases. When these enzymes cleave the dye-substrate complex, a fluorescence signal is emitted, indicating enzyme activity.

Azo dyes exhibit vibrant colors and can change color in response to enzymatic activity. Enzymes such as laccases and peroxidases can break down azo dyes, leading to a visible color change. This property is utilized in bioremediation processes to monitor the degradation of pollutants.

Peptide-Based Dyes are engineered to include specific peptide sequences recognized by target proteases. When the protease cleaves the peptide, the dye undergoes a structural change, resulting in a visible color shift. This approach is useful for detecting protease activity in various biological samples.

Ester-Containing Dyes with ester bonds can be cleaved by lipases, leading to a color change. This property is particularly useful in monitoring lipase activity in conditions such as pancreatitis.

It is an object of the present invention to provide a sensitive, accurate, and rapid method usable in any patient care environment for identification of pathogens in bodily fluids at critical thresholds of presence before clinical signs and symptoms of infection present.

It is an object of the present invention to obviate at least one disadvantage of previous methods intended for identification of pathogen presence in fluids, including at least providing universal connectivity in standard catheter and fluid extraction devices, detection of multiple pathogens, and a rapid response observable alert independent of a host response.

It is an object of the present invention to utilize biomarkers to detect pathogens in real time, where biomarkers are characteristics objectively measured as indicators of health, disease, or a response to an exposure or intervention, including therapeutic interventions.

In one aspect, the present invention provides a fluid monitoring device comprising a capsule containing a biosensor responsive to the presence of at least one pathogen specific biomarker, the biosensor adapted to provide a visual indication when the biomarker is present in a fluid exposed to the biosensor in the capsule, no wires, batteries, or external devices needed.

In another aspect, the capsule in the fluid monitor includes a first orifice and a second orifice, and each orifice is adapted to connect to a catheter lumen and allow passage of a fluid.

In another aspect, the first orifice and said second orifice of the capsule are each adapted to connect to a conduit adapted to transport a fluid.

In another aspect, the first orifice of the capsule is adapted to connect to a conduit adapted to transport a fluid from a catheter.

In another aspect, the second orifice of the capsule is adapted to transport a fluid from the capsule toward an open or closed collector.

In another aspect, the first orifice and said second orifice of the capsule are each adapted to receive universal connectors to connect to a conduit adapted to transport a fluid.

In another aspect, the capsule is adapted to enable visual inspection of the biosensor.

In another aspect, the capsule body is transparent medical grade plastic to enable visual inspection of the biosensor.

In another aspect, the capsule body may be configured with transparent viewing port to enable visual inspection of the biosensor if the capsule body is configured in opaque material.

In another aspect, the capsule body may comprise any of Polycarbonate, Polysulfone, Polyethylene terephthalate (PET), and analogs thereof.

In another aspect, the biosensor comprises a nanofiber membrane adapted to exhibit a color-change response to the presence of at least one biomarker.

In another aspect, the biosensor comprises a nanofiber membrane electrospun using fiberizeable material formed in at least three adjacent layers of nanofibers, each layer comprising a plurality of aligned nanofibers oriented at oblique angles relative to nanofibers in adjacent layers, said plurality of aligned nanofibers in each layer extending from a first membrane edge to a second membrane edge.

In another aspect, the biosensor membrane comprises a plurality of aligned nanofibers in each layer cross a plurality of aligned nanofibers in adjacent layers at a plurality of distinct points of intersection, forming a crossing at each distinct point of intersection consisting of three crossing nanofibers extending directionally as six radials from each of said distinct points of intersection.

In another aspect, the biosensor membrane comprises a plurality of aligned nanofibers exhibiting relative cross-alignment angles between six radials at distinct points of intersection in the range of 50 to 70 degrees, and the relative cross-alignment angles of the aligned nanofibers and spacing between the aligned nanofibers each have a specific average numerical value in the membrane.

In another aspect, the biosensor comprises nanofibers further selected from any one or combination of poly (lactic-co-glycolic acid) (PLGA), polyvinylpyrrolidone (PVP), poly(ethyleneoxide) (PEO), polyuretha ne (PU), PVP/cyclodextrin, polyvinyl alcohol (PVA), polycaprolactone (PCL), cellulose, PVP/ethyl cellulose, PVP/zein, cellulose acetate, hydroxypropyl methylcellulose (HPMC), and analogues thereof.

In one aspect, a method of manufacturing a fluid monitor is provided, the method including obtaining a nanofiber membrane adapted as a biosensor to exhibit a color-change response to the presence of at least one biomarker specific to pathogens; obtaining a capsule having a first orifice and a second orifice each adaptable to connect to conduits (i.e., lumen) typically used in any of urinary catheters, chest tubes, wound drains, endotracheal tubes, central lines, hemodialysis ports, surgical site drains, and bypass grafts, the capsule comprising a transparent enclosure or a transparent port enabling visual inspection of an enclosed biosensor; positioning within the capsule the nanofiber membrane oriented for exposure to a fluid when the fluid is present within the capsule.

In another aspect, the biosensor may be fabricated by electospinning nanofiber to produce a nanofiber structure directly on to a fiber collector pallet. In a preferred embodiment, the biosensor may be fabricated according to the methods and disclosures of U.S. Pat. No. 11,208,735 by the same inventor hereof. The structure of the biosensor may comprise at least a plurality of aligned nanofibers in each of multiple layers (at least three) that cross a plurality of aligned nanofibers in adjacent layers at a plurality of distinct points of intersection, forming a crossing at each distinct point of intersection consisting of three crossing nanofibers extending directionally as six radials from each distinct point of intersection, where a plurality of nanofibers in each layer of the biosensor cross at relative cross-alignment angles between six radials at each distinct point of intersection in the range of 50 to 70 degrees.

In one aspect, the biosensor may be fabricated as an integrated component by electospinning nanofiber directly on to an absorbent fabric as a collector pallet to which fibers attach according to the disclosures of U.S. Pat. No. 11,208,735 by the same inventor hereof.

In another aspect, the absorbent layer may comprise natural or synthetic material.

In another aspect, the absorbent layer may comprise natural or synthetic material infused with a neutralizing agent that may include any of acetic acid, citric acid, hypochlorous acid, and analogues thereof.

In another aspect, the capsule may further comprise any of an absorbent or fluid sampling reservoir.

In another aspect, the fluid sampling reservoir may comprise any of diverters, baffles, and microfluidic channels through which fluids flow and are exposed to the biosensor.

In another aspect, a flow diverter may incorporate a filtration membrane intended to reduce salts and other contaminants in a fluid to be sampled.

In one aspect, a method is provided for indicating pathogen presence in a fluid, the method including obtaining a capsule encapsulating a biosensor that exhibits a color-change response to the presence of at least one pathogen specific biomarker; connecting a first orifice present on the capsule to a first conduit; connecting a second orifice present on the capsule to a second conduit; passing a fluid through the first orifice into the capsule from the first conduit and out of the second orifice into the second conduit; wherein the biosensor is exposed to the fluid passing through the capsule, and any color-change response is observable.

In another aspect, the capsule is inserted in-line and connected between a catheter and a fluid collection container in a urinary catheter device or a passive surgical site drainage device.

In another aspect, the capsule is inserted in-line between a catheter and an open or closed fluid collection container in a urinary catheter device.

In another aspect, the capsule is inserted in-line between a catheter and a closed fluid collection container in an indwelling urinary catheter device.

In another aspect, the capsule is inserted in-line between a catheter intended for intermittent use and an open fluid collection container.

In another aspect, a first segment of tubing is connected to a catheter a conduit, and a second segment of tubing connected to the collection container as a second conduit, where the catheter is configured to drain urine from a bladder or fluid from a surgical site.

In another aspect, the capsule is inserted in-line upstream or downstream of a suction pump delivering a fluid to a collection container in a negative pressure therapy device, the device configured to extract exudate from a wound or fluid from a surgical site.

In another aspect, a first conduit is a segment of hollow tubing (i.e. lumen) connected to a suction pump and a second conduit is a segment of hollow tubing connected to a collection vessel.

In another aspect, the capsule is connected in-line to a catheter in a urinary catheter device or a passive surgical site drainage device where fluid passes though the capsule toward an open catchment.

In another aspect, the biosensor comprises any of means of exhibiting response to the presence of said at least one biomarker, including built-in electrical sensors, volatile organic compound sniffers, chemical detectors, photonics, pH sensors, and the like.

In another aspect, the biosensor comprises a nanofiber membrane adapted to exhibit a color-change response to the presence of at least one biomarker.

In another aspect, the biomarker is any of protease and lipase.

In another aspect, the biosensor incorporates an organic dye.

In another aspect, the biosensor incorporates an organic dye that exhibits a color-change response when exposed to any of protease and lipase.

In another aspect, the biosensor incorporates an organic dye selected from any of a peptide-based dye and an ester-containing dye.

In another aspect, the biosensor incorporates an organic dye that exhibits a color-change response when exposed to pancreatic lipase.

In another aspect, the biosensor further comprises polyurethane (PU) core-shell nanofiber, and a hemicyanine-based chromogenic probe surface localized in the core-shell nanofiber.

In another aspect, the hemicyanine-based chromogenic probe further comprises a labile ester linkage that is enzymatically cleavable by lipase released from clinically relevant strains of bacteria and fungi.

In another aspect, the biosensor may incorporate an organic dye responsive to enzymes secreted by various concentrations of ESKAPEE bacteria (E. faecium, S. aureus, K. pneumoniae, A. baumannii, P. aeruginosa, Enterobacter species, and E. coli) Candida spp. (e.g., C. auris, C. Albicans).

### BRIEF DESCRIPTION OF THE DRAWINGS

Embodiments of the present disclosure will now be described, by way of example only, with reference to the attached Figures. The components, their geometries and configurations shown are illustrative and not to be construed as limiting.

FIG. 1 shows a non-limiting diagram of the capsule of the present invention.

FIG. 2 shows a non-limiting diagram of the biosensor positioned in the capsule of the present invention.

FIG. 3 shows a non-limiting diagram of the capsule of the present invention including a sampling reservoir holding the biosensor.

FIG. 4 shows a non-limiting diagram of the capsule of the present invention including a sampling absorbent and the biosensor in the sampling reservoir.

FIG. 5 shows a non-limiting diagram of the capsule of the present invention including a flow diverter and the biosensor in the sampling reservoir.

FIG. 6A shows a non-limiting diagram of the capsule of the present invention connected in a conduit (i.e., lumen) of a closed urinary catheter device or passive surgical site drainage device.

FIG. 6B shows a non-limiting diagram of the capsule of the present invention connected in a conduit (i.e., lumen) of an open urinary catheter device or passive surgical site drainage device.

FIG. 7 shows a non-limiting diagram of the capsule of the present invention in a conduit upstream from a pump in a negative pressure device for extracting fluid from a wound or surgical site.

FIG. 7A shows a non-limiting diagram of the capsule of the fluid monitoring device of the present invention positioned between a conduit (i.e., lumen) and upstream from a pump in a negative pressure device for extracting fluid from a wound or surgical site, and positioned for connection to a wound connector placed on an absorbent material intended to cover a wound.

FIG. 8 shows a non-limiting diagram of the capsule of the present invention in a conduit downstream from a pump in a negative pressure device for extracting fluid from a wound or surgical site.

FIG. 9 shows a non-limiting diagram of the capsule of the present invention in the shape of a rectangle, where a first part of the outer portion of the capsule is joined by a second part of the outer portion of the capsule at a middle seam.

FIG. 10 shows a non-limiting diagram of the capsule of the present invention in the shape of a cylinder, where a first part of the outer portion of the capsule is joined by a second part of the outer portion of the capsule at a middle seam.

FIG. 11 shows a non-limiting diagram of the capsule of the present invention in the shape of a rectangle, where a first part of the outer portion of the capsule is a top portion of the rectangle joined by a second part of the outer portion of the capsule that is a bottom portion of the rectangle at a middle seam.

FIG. 12 shows a non-limiting diagram of an exploded view of one embodiment of the present invention.

### DETAILED DESCRIPTION OF PREFERRED EMBODIMENTS OF THE INVENTION

### In Brief

Preferred embodiments of the present disclosure will now be described with reference to the attached Figures. The components, their geometries and configurations shown are illustrative and not to be construed as limiting.

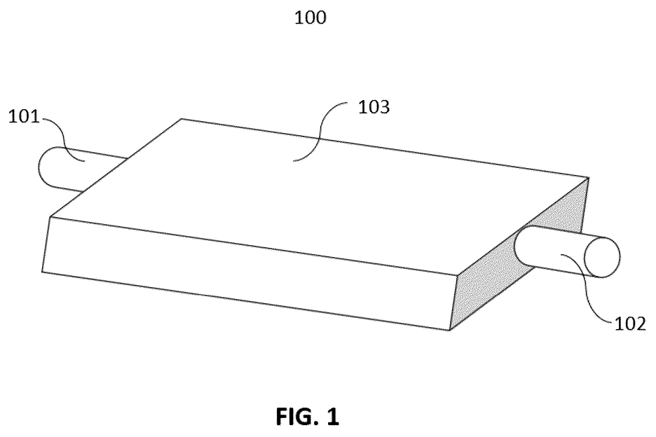

FIG. 1 shows a non-limiting diagram of the capsule of the fluid monitoring device of the present invention comprising a biosensor, the capsule adapted to contain the biosensor, and further adapted to present at least a visual indication when a biomarker is present in a fluid exposed to the biosensor in the capsule of the fluid monitoring device. The capsule may comprise a transparent medical grade plastic or be configured with a transparent viewing port if opaque material is used. The capsule body may comprise any of Polycarbonate, Polysulfone, Polyethylene terephthalate (PET), and analogs thereof. The capsule is volumetrically sized to allow full flow of fluid from a first conduit and into a second conduit without backup into the first conduit. The biosensor can continuously monitor a fluid flowing though the capsule.

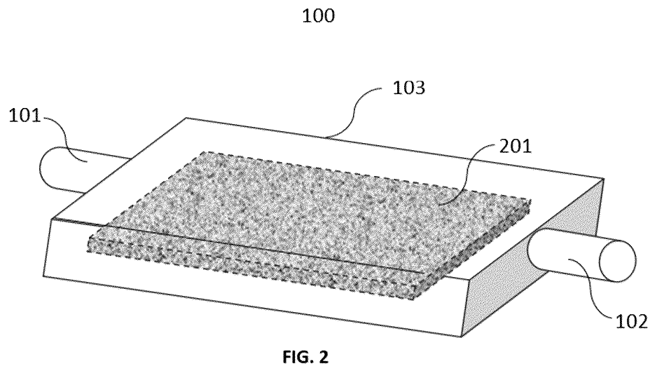

FIG. 2 shows a non-limiting diagram of the biosensor positioned in the capsule the fluid monitoring device of the present invention. A biosensor is held in position for exposure to fluid flowing through a first orifice into the capsule and out of a second orifice. The biosensor is responsive to a least one pathogen specific biomarker, and comprises any of means of detecting and exhibiting response to the presence of at least one biomarker. The biosensor may comprise at least a plurality of aligned nanofibers in each of multiple layers that cross a plurality of aligned nanofibers in adjacent layers at a plurality of distinct points of intersection, forming a crossing at each distinct point of intersection consisting of three crossing nanofibers extending directionally as six radials from each distinct point of intersection, where a plurality of nanofibers in each layer of the biosensor cross at relative cross-alignment angles between six radials at each distinct point of intersection in the range of 50 to 70 degrees. The nanofiber membrane may incorporate an organic dye responsive to a pathogen produced enzyme. Further, a chromogenic probe comprising a bioactive dye such as but not limited to a hemicyanine-based dye may be included in the nanofiber membrane. A hemicyanine-based dye comprising a labile ester linkage is enzymatically cleavable by a biomarker released from clinically relevant strains of bacteria and fungi. The biosensor may be adapted to exhibit a color-change response to the presence of at least one biomarker, where the biomarker is a pathogen produced enzyme. The pathogen produced enzymes may include at least one of protease and lipase. The organic dye may be selected to exhibit a color-change from yellow to green, purple to red, blue to red or other distinctly contrasting colors in response to enzymes secreted by clinically relevant pathogens. The biosensor may exhibit an initial color such as yellow and transition to a different color such as green or red when exposed to pathogen produced enzymes.

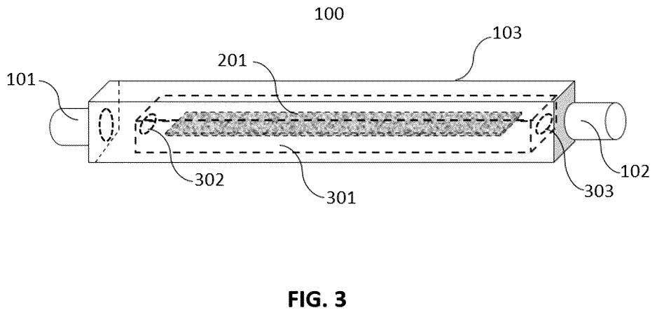

FIG. 3 shows a non-limiting diagram of the capsule the fluid monitoring device of the present invention including a sampling reservoir holding the biosensor. The sampling reservoir is purposed to temporarily retain fluid for a period of time sufficient for the biosensor to respond to the presence of a biomarker in the fluid passing through the capsule.

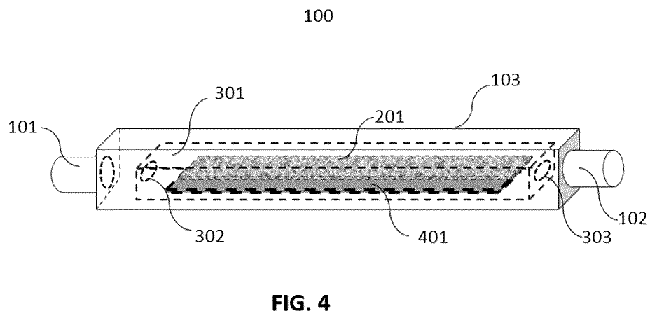

FIG. 4 shows a non-limiting diagram of the capsule the fluid monitoring device of the present invention including a sampling absorbent and the biosensor in the sampling reservoir. The absorbent in the sampling reservoir is purposed to aid in temporarily retaining fluid for a period of time sufficient for the biosensor to respond to the presence of a biomarker in the fluid passing through the capsule. The absorbent layer may comprise natural or synthetic material, and may be infused with a neutralizing agent that may include any of acetic acid, citric acid, hypochlorous acid, and analogues thereof.

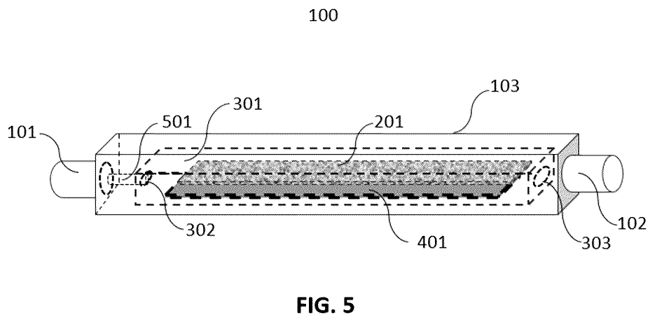

FIG. 5 shows a non-limiting diagram of the capsule the fluid monitoring device of the present invention including a flow diverter and the biosensor in the sampling reservoir. Including a flow diverter in the sampling reservoir is purposed to control the flow rate of fluid through the capsule and aid in temporarily retaining fluid for a period of time sufficient for the biosensor to respond to the presence of a biomarker in the fluid passing through the capsule. The flow diverter may incorporate a filtration membrane intended to reduce salts and other contaminants in the fluid to be sampled. The filtration membrane may allow some salts and contaminants to pass through while still removing larger molecules. A neutralizing agent such as citric acid and analogues thereof may be incorporated in the filtration membrane to alter the pH of the fluid to be sampled.

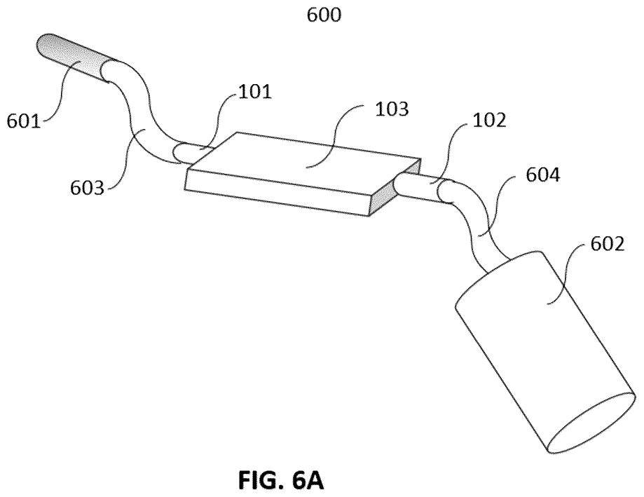

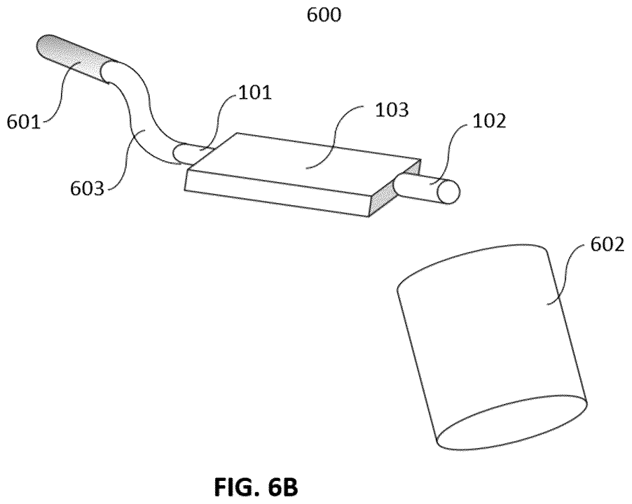

FIG. 6A shows a non-limiting diagram of the capsule the fluid monitoring device of the present invention connected in a conduit of a closed urinary catheter device or passive surgical site drainage device. Fluid draining from a catheter connected conduit flows through the capsule of the present invention and is exposed to a biosensor, thereafter exiting into a connected catchment. The closed device is typical of those used with an indwelling catheter.

FIG. 6B shows a non-limiting diagram of the capsule the fluid monitoring device of the present invention connected in a conduit of an open urinary catheter device or passive surgical site drainage device. Fluid draining from a catheter connected conduit flows through the capsule of the present invention and is exposed to a biosensor, thereafter exiting and flowing to a catchment. The open device is typical of those used with intermittent urinary catheterization with discharge to a toilet or potable container, or an indwelling surgical drain where fluid is discharged to an absorbent such as gauze positioned on the skin surface of a patient.

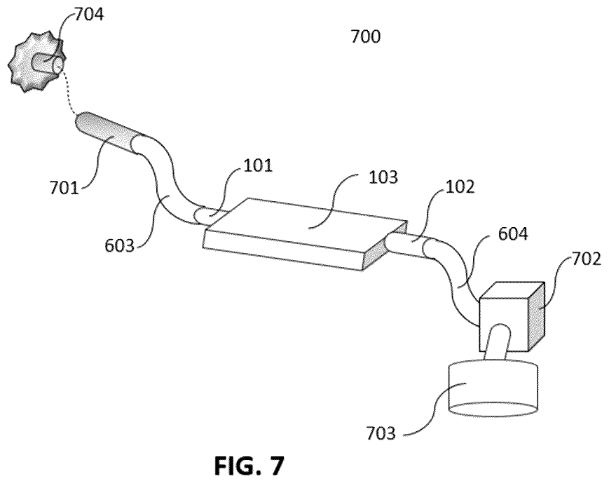

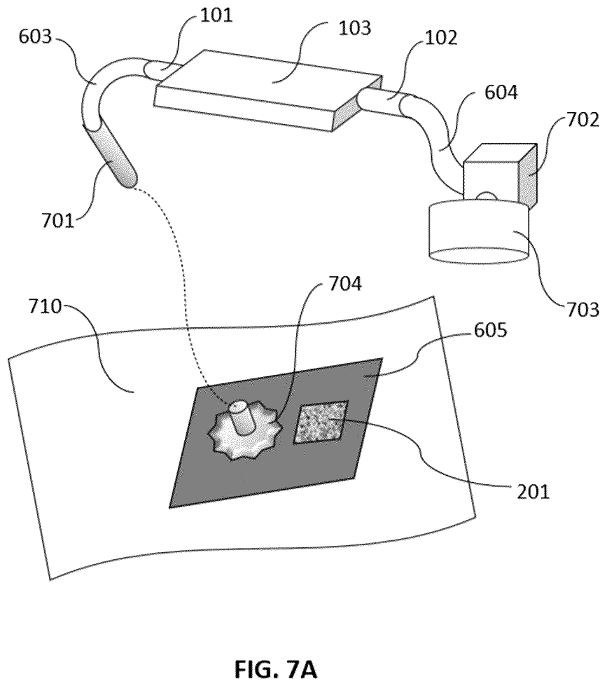

FIG. 7 shows a non-limiting diagram of the capsule of the fluid monitoring device of the present invention in a conduit upstream from a pump in a negative pressure device for extracting fluid from a wound or surgical site. Fluid drawn by a negative pressure exerted by a pump is drawn through a conduit attached to the capsule of the present invention, where the fluid flows through the capsule and is exposed to a biosensor. The fluid flowing through the capsule is drawn through the conduit by the pump and into a connected catchment.

FIG. 7A shows a non-limiting diagram of the capsule of the fluid monitoring device of the present invention positioned between a conduit and upstream from a pump in a negative pressure device for extracting fluid from a wound or surgical site, and positioned for connection to a wound connector placed on an absorbent material intended to cover a wound. An additional biosensor as is used in the fluid monitoring device of the present invention may also be placed onto the absorbent material before covering the absorbent material and a wound attachment with a transparent adhesive drape. As wound fluid is absorbed into the absorbent material, the biosensor will be exposed to contact with the wound fluid absorbed. The biosensor may exhibit an initial color such as yellow and transition to a different color such as green or red when exposed to pathogen produced enzymes present in the wound fluid.

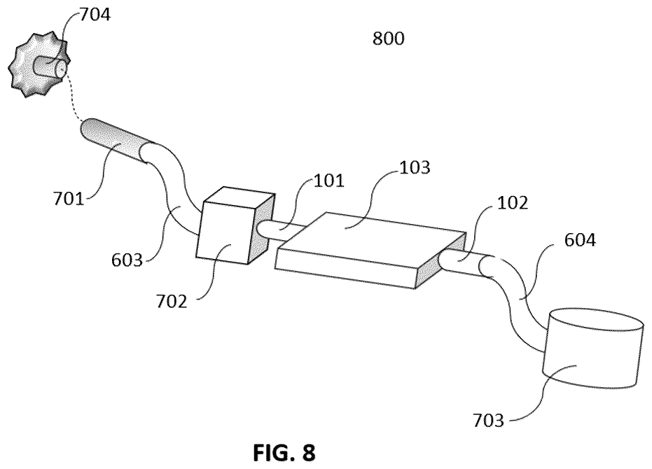

FIG. 8 shows a non-limiting diagram of the capsule the fluid monitoring device of the present invention in a conduit downstream from a pump in a negative pressure device for extracting fluid from a wound or surgical site. Fluid drawn by a negative pressure exerted by a pump is drawn through a conduit attached to the pump and pushed into the capsule of the present invention, where the fluid flows through the capsule and is exposed to a biosensor.

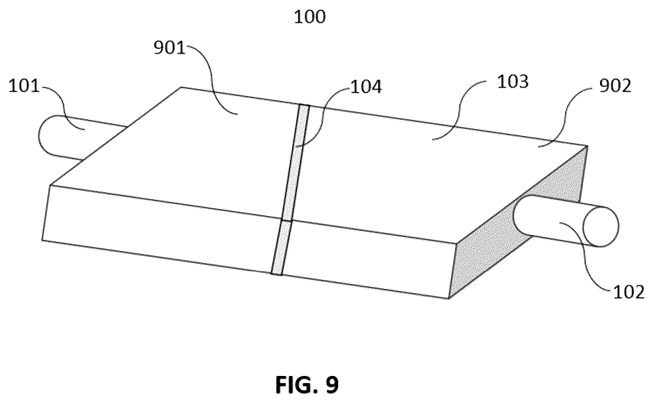

FIG. 9 shows a non-limiting diagram of the capsule the fluid monitoring device of the present invention in the shape of a rectangle, where a first part of the outer portion of the capsule is joined by a second part of the outer portion of the capsule at a middle seam. The two-part structure enables insertion of the biosensor before the two parts are joined to form a leak-proof seal.

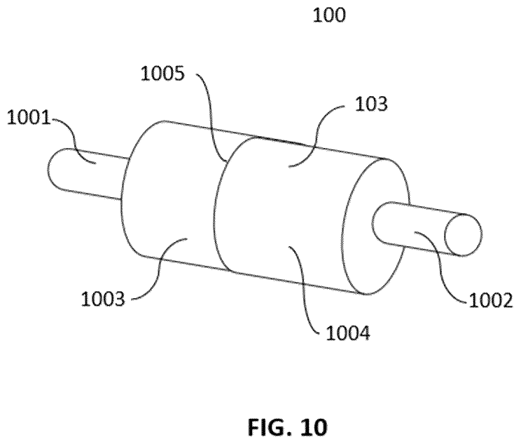

FIG. 10 shows a non-limiting diagram of the capsule the fluid monitoring device of the present invention in the shape of a cylinder, where a first part of the outer portion of the capsule is joined by a second part of the outer portion of the capsule at a middle seam. The two-part structure enables insertion of the biosensor before the two parts are joined to form a leak-proof seal.

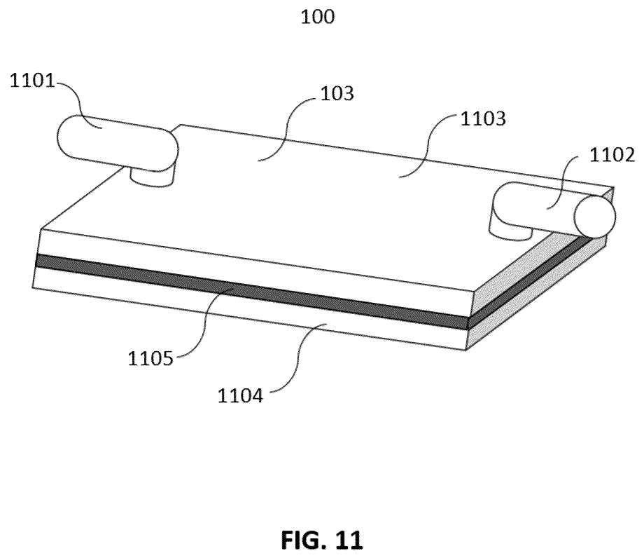

FIG. 11 shows a non-limiting diagram of the capsule of the fluid monitoring device of the present invention in the shape of a rectangle, where a first part of the outer portion of the capsule is a top portion of the rectangle joined by a second part of the outer portion of the capsule that is a bottom portion of the rectangle at a middle seam. The two-part structure enables insertion of the biosensor before the two parts are joined to form a leak-proof seal.

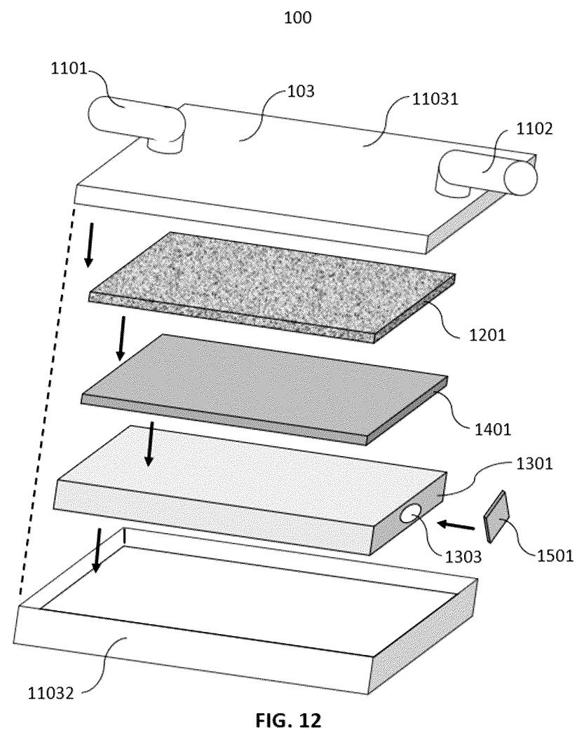

FIG. 12 shows a non-limiting diagram of an exploded view of a preferred embodiment the fluid monitoring device of the present invention in the shape of a rectangle. Component parts comprising the present invention are shown in relation to how component parts may be assembled. In a preferred embodiment, the biosensor may be fabricated by electospinning nanofiber to produce a nanofiber structure directly on to a fiber collector pallet according to the methods and disclosures of U.S. Pat. No. 11,208,735 by the same inventor hereof. The structure of the biosensor may comprise at least a plurality of aligned nanofibers in each of multiple layers (at least three) that cross a plurality of aligned nanofibers in adjacent layers at a plurality of distinct points of intersection, forming a crossing at each distinct point of intersection consisting of three crossing nanofibers extending directionally as six radials from each distinct point of intersection, where a plurality of nanofibers in each layer of the biosensor cross at relative cross-alignment angles between six radials at each distinct point of intersection in the range of 50 to 70 degrees. In another preferred embodiment, the biosensor may be fabricated as an integrated component by electospinning nanofiber directly on to an absorbent fabric as a fiber collector pallet according to the disclosures of U.S. Pat. No. 11,208,735 by the same inventor hereof.

### In Detail

Referring now to FIG. 1, a non-limiting diagram shows the fluid monitoring device 100 of the present invention, the invention comprising a biosensor (FIG. 2, 201) and a capsule 103 adapted to contain the biosensor (FIG. 2, 201), where the biosensor (FIG. 2, 201) is adapted to provide at least a visual indication when a biomarker is present in a fluid exposed to the biosensor (FIG. 2, 201) in the capsule 103 of the fluid monitoring device 100. The capsule 103 may be a transparent medical grade plastic or be configured with a transparent viewing port (not shown) if opaque material is used. The capsule 103 may comprise any of Polycarbonate, Polysulfone, Polyethylene terephthalate (PET), and analogs thereof. The capsule 103 as shown is rectangular, however, any effective shape may be used. The capsule 103 is sized volumetrically to allow full flow of fluid from a first conduit 101 and into a second conduit 102 without backup into the first conduit 101.

FIG. 2 shows a non-limiting diagram of a biosensor 201 positioned in the capsule 103 of the fluid monitoring device 100 of the present invention. The biosensor 201 is held in position for exposure to fluid flowing through a first orifice 101 into the capsule 103 and out of a second orifice 102. The biosensor 201 is responsive to a least one pathogen produced biomarker, and comprises any of means of detecting and exhibiting response to the presence of at least one biomarker, including any enzyme secreted by pathogenic bacteria and fungus. The biosensor 201 may comprise a membrane including at least a plurality of aligned nanofibers in each of multiple layers that cross a plurality of aligned nanofibers in adjacent layers at a plurality of distinct points of intersection, forming a crossing at each distinct point of intersection consisting of three crossing nanofibers extending directionally as six radials from each distinct point of intersection, where a plurality of nanofibers in each layer of the biosensor 201 cross at relative cross-alignment angles between six radials at each distinct point of intersection in the range of 50 to 70 degrees. The membrane may incorporate an organic dye responsive to a pathogen produced enzyme. Further, a chromogenic probe comprising a bioactive dye such as but not limited to a hemicyanine-based dye and may be included in the membrane. A hemicyanine-based dye comprising a labile ester linkage is enzymatically cleavable by a biomarker released from clinically relevant strains of bacteria and fungi. The biosensor 201 may be adapted to exhibit a color-change response to the presence of at least one biomarker, where the biomarker is a pathogen produced enzyme. The pathogen produced enzymes may include at least one of protease and lipase. The organic dye may be selected to exhibit a color-change from yellow to green, purple to red, blue to red or other distinctly contrasting colors in response to enzymes secreted by clinically relevant pathogens. The biosensor 201 may exhibit an initial color such as yellow and transition to a different color such as green or red when exposed to pathogen produced enzymes, independent of a host response (e.g., elevated pH, clinical signs and symptoms).

FIG. 3 shows a non-limiting diagram of the capsule 103 of the fluid monitoring device 100 of the present invention including a sampling reservoir 301 holding the biosensor 201. The sampling reservoir 301 is purposed to temporarily retain fluid for a period of time sufficient for the biosensor 201 to respond to the presence of a biomarker in the fluid passing through the capsule 103 and entering by way of the first orifice 101. A portion of the fluid flowing though the capsule 103 enters the reservoir 301 by way of a first port 302 where the biosensor 201 is exposed to the held fluid. The sampled fluid exits the reservoir 301 by way of a second port 303 comingling with fluid exiting the capsule 103 though the second orifice 102.

FIG. 4 shows a non-limiting diagram of the capsule 103 of the fluid monitoring device 100 of the present invention including a sampling absorbent 401 and the biosensor 201 in the sampling reservoir 301. The absorbent 401 in the sampling reservoir 301 is purposed to aid in temporarily retaining fluid for a period of time sufficient for the biosensor 201 to respond to the presence of a biomarker in the fluid passing through the capsule 103. In one preferred embodiment, the sampling reservoir 301 is configured with a first port 302 and a second port 303 to limit fluid flow into and out of the sampling reservoir 301. The absorbent 401 may comprise natural or synthetic material. The absorbent layer 401 may comprise natural or synthetic materialinfused with a neutralizing agent that may include any of acetic acid, citric acid, hypochlorous acid, and analogues thereof.

FIG. 5 shows a non-limiting diagram of the capsule 103 of the fluid monitoring device 100 of the present invention including a flow diverter 501 and the biosensor 201 in the sampling reservoir 301. Including a flow diverter 501 in the sampling reservoir 301 is purposed to control the flow rate of fluid through the capsule 103 and aid in temporarily retaining fluid for a period of time sufficient for the biosensor 201 to respond to the presence of a biomarker in the fluid passing through the capsule 103. The flow diverter 501 may incorporate a filtration membrane (not shown) intended to reduce salts and contaminants in the fluid to be sampled. The composition of the filtration membrane (not shown) may allow some salts to pass through while still removing larger molecules. A neutralizing agent may be incorporated in the filtration membrane (not shown) to alter the pH of the fluid to be sampled.

FIG. 6A shows a non-limiting diagram of the capsule 103 of the fluid monitoring device (FIG. 1, 100) of the present invention connected in a conduit 603 of a fluid drainage system 600 configured as a closed system applicable to use as an indwelling urinary catheter or a passive surgical site drain. Fluid draining from a catheter 601 into the connected conduit 603 flows through the first orifice 101 into the capsule 103 of the fluid monitoring device (FIG. 1, 100) and is exposed to a biosensor (FIG. 2, 201), the fluid thereafter exiting though the second orifice 102 into a conduit 604 connected to a catchment 602. The biosensor (FIG. 2, 201) visible though the transparent material comprising the capsule 103 or a viewing port (not shown) may exhibit an initial color such as yellow and transition to a different color such as green or red when exposed to pathogen produced enzymes present in the wound fluid.

FIG. 6B shows a non-limiting diagram of the capsule 103 of the fluid monitoring device (FIG. 1, 100) of the present invention connected in a conduit 603 of a fluid drainage system 600 configured as an open system applicable to use as an intermittent urinary catheter or passive surgical site drain. Fluid draining from a catheter 601 connected conduit 603 flows through the first orifice 101 into the capsule 103 of the fluid monitoring device (FIG. 1, 100) of the present invention and is exposed to a biosensor (FIG. 2, 201), the fluid thereafter exiting through the second orifice 102 into a catchment 602. The open configuration may be used for intermittent urinary catheterization with discharge to a catchment 602 such as a toilet or potable container, or for an indwelling surgical drain where fluid is discharged to an absorbent catchment 602 such as a gauze pad positioned on the skin surface of a patient. The biosensor (FIG. 2, 201) visible though the transparent material comprising the capsule 103 or a viewing port (not shown) may exhibit an initial color such as yellow and transition to a different color such as green or red when exposed to pathogen produced enzymes present in the wound fluid.

FIG. 7 shows a non-limiting diagram of the capsule 103 of the fluid monitoring device (FIG. 1, 100) of the present invention positioned between a conduit 603 and 604 upstream from a pump 702 in a negative pressure device 700 for extracting fluid from a wound or surgical site. Fluid drawn by a negative pressure exerted by a pump 702 is drawn from a wound attachment 704 through a conduit 701 and 603 and flows through the first orifice 101 into the capsule 103 of the fluid monitoring device (FIG. 1, 100), where the fluid flows through the capsule 103 and is exposed to a biosensor (FIG. 2, 201) and drawn through the second orifice 102 and conduit 604 by the pump 702 and into a connected catchment 703. The biosensor (FIG. 2, 201) visible though the transparent material comprising the capsule 103 or a viewing port (not shown) may exhibit an initial color such as yellow and transition to a different color such as green or red when exposed to pathogen produced enzymes present in the wound fluid.

FIG. 7A shows a non-limiting diagram of the capsule 103 of the fluid monitoring device (FIG. 1, 100) of the present invention positioned between a conduit 603 and 604 upstream from a pump 702 in a negative pressure device 700 for extracting fluid from a wound or surgical site, and positioned for connection to a wound connector 704 placed on an absorbent material 605 intended to cover a wound (not shown). Some negative pressure devices utilize an absorbent material 605 (e.g., a foam) placed onto a wound to absorb wound exudate. A wound attachment 704 may be placed onto the absorbent material 605 covered with a transparent adhesive drape (not shown) that secures the absorbent material 605 to the skin 710 surrounding a wound. The drape is punctured and the wound attachment 704 is adhered to the drape over the puncture site. The biosensor (FIG. 2, 201) visible though the transparent material comprising the capsule 103 or a viewing port (not shown) may exhibit an initial color such as yellow and transition to a different color such as green or red when exposed to pathogen produced enzymes present in the wound fluid. An additional biosensor 201 as is used in the fluid monitoring device (FIG. 2, 100) of the present invention can be placed onto the absorbent material 605 before covering the absorbent material 605 and wound attachment 704 with an additional transparent adhesive drape (not shown). As wound fluid is absorbed into the absorbent material, the additional biosensor 201 will be exposed to contact with the wound fluid absorbed. The additional biosensor 201 may exhibit an initial color such as yellow and transition to a different color such as green or red when exposed to pathogen produced enzymes present in the wound fluid.

FIG. 8 shows a non-limiting diagram of the capsule 103 of the fluid monitoring device (FIG. 1, 100) of the present invention positioned between conduit 603 and 604 downstream from a pump 702 in a negative pressure device 800 for extracting fluid from a wound or surgical site. Fluid drawn by a negative pressure exerted by a pump 702 is drawn through a conduit 603 attached to the pump 702 and pushed into the capsule 103 though the first orifice 101 of the fluid monitoring device (FIG. 1, 100), where the fluid flows through the capsule 103 and is exposed to a biosensor (FIG. 2, 201) and thereafter exits though through the second orifice 102 and conduit 604 into a connected catchment 703. The biosensor (FIG. 2, 201) visible though the transparent material comprising the capsule 103 or a viewing port (not shown) may exhibit an initial color such as yellow and transition to a different color such as green or red when exposed to pathogen produced enzymes present in the wound fluid.

FIG. 9 shows a non-limiting diagram of the capsule (FIG. 1, 103) of the fluid monitoring device (FIG. 1, 100) of the present invention in the shape of a rectangle, where a first part 901 of the outer portion of the capsule 103 is joined by a second part 902 of the outer portion of the capsule 103 at a middle seam 104. The two-part structure enables insertion of the biosensor (FIG. 2, 201) before the two parts 901 and 902 are joined to form a leak-proof seal at the middle seam 104. Fluid flows into the capsule (FIG. 1, 103) though first orifice 101 and thereafter exits though the second orifice 102.

FIG. 10 shows a non-limiting diagram of the capsule 103 of the fluid monitoring device (FIG. 1, 100) of the present invention in the shape of a cylinder, where a first part 1003 of the outer portion of the capsule 103 is joined at a middle seam 1005 by a second part 1004. The two-part structure enables insertion of the biosensor (FIG. 2, 201) before the two parts 1003 and 1004 are joined to form a leak-proof seal at the middle seam 1005. Fluid flows into the capsule 103 though first orifice 1001 and thereafter exits though the second orifice 1002.

FIG. 11 shows a non-limiting diagram of the capsule 103 of the fluid monitoring device 100 of the present invention in the shape of a rectangle, where a first part 1103 of the outer portion of the capsule 103 is a top portion of the rectangle joined by a second part 1104 of the outer portion of the capsule 103 that is a bottom portion of the rectangle connected at a middle seam 1105. The two-part structure enables insertion of the biosensor (FIG. 2, 201) before the two parts 1103 and 1104 are joined to form a leak-proof seal at the middle seam 1105. The first orifice 1101 and the second orifice 1102 are connected at the top surface of the first portion 1103. Fluid flows into the capsule 103 though first orifice 1001 and thereafter exits though the second orifice 1002.

FIG. 12 shows a non-limiting diagram of an exploded view of a preferred embodiment of the present invention 100 in the shape of a rectangle. Component parts comprising the present invention 100 are shown in relation to how component parts may be assembled. The internal components of the capsule 103 may comprise any of an absorbent 1401, a flow diverter 1501, and fluid sampling reservoir 1301. Including a sampling absorbent 1401 and the biosensor 1201 in the sampling reservoir 1301 may be needed for some applications, but not all. The absorbent 1401 in the sampling reservoir 1301 is purposed to aid in temporarily retaining fluid for a period sufficient for the biosensor 1201 to respond to the presence of a biomarker in the fluid passing through the capsule 103. The exploded view of a notional capsule 103 is shown configured with multiple components, where the top 11031 and bottom 11032 portions of the capsule 103 hold the internal components, comprising the biosensor 1201, the absorbent 1401 and the sampling reservoir 1301. A diverter 1501 may be needed to alter flow into the sampling reservoir through an orifice 1303, and in some embodiments a filtration membrane (not shown) may be included. Fluid flows into the capsule 103 though first orifice 1001 and thereafter exits though the second orifice 1002.

In a non-limiting method enabling fabrication of at least one capsule 103 of the fluid monitoring device (FIG. 1, 100) of the present invention comprising parts as shown in FIG. 12, the outer portion of the capsule 103 may be assembled from two parts 11031 and 11032 that may be injection molded using a medical grade polymer. The inner components may include a sampling reservoir 1301 that also may be injection molded from a transparent medical grade polymer. The capsule parts 11031 and 11032, and the sampling reservoir 1301 may comprise any of Polycarbonate, Polysulfone, Polyethylene terephthalate (PET), and analogs thereof. The outer portion parts 11031 and 11032, and sampling reservoir 1301 may also be fabricated using additive manufacturing methods (e.g., 3D printing). The capsule 103 outer portion parts 11031 and 11032 may be fabricated to include retention elements designed to hold a biosensor 1201 or the combined biosensor 1201 and sampling reservoir 1301 in position within the capsule 103. The sampling reservoir 1301, or one or both outer portions of the capsule 103, may be fabricated with at least one flow diverter 1501 to control the direction and rate of flow of a fluid over the biosensor 1201 which may be enclosed within a sampling reservoir 1301. An absorbent 1401 may also be enclosed within the sampling reservoir 1301. The biosensor 1201 of the fluid monitoring device (FIG. 1, 100) of the present invention may be placed in the sampling reservoir when the reservoir is intended to be included as a part of the configured capsule (FIG. 1, 100). The biosensor 1201 or the biosensor 1201 held in a sampling reservoir 1301 is inserted into one of the capsule 103 outer parts 11031 or 11032 and the first part 11031 is engaged with the opposite second part 11032.

The two outer parts 11031 and 11032 of the capsule 103 of the fluid monitoring device (FIG. 1, 100) of the present invention may be joined together using at least two alternative methods. The first part 11031 and the second part 11032 may be fabricated to include a snap ridge or screw threads when a cylindrical shape is used that form a leak-proof seal when the two parts 11031 and 11032 are joined together. Alternatively, the two outer parts 11031 and 11032 of the capsule may be welded together where the first part 11031 engages at a middle seam the second part 11032. Various seam welding machines are commercially available, including at least ultrasonic welding machines, infrared welding machines, and hot air welding machines. The machine selected for welding a middle seam shown in FIGS. 9, 10, and 11 of the capsule 103 of the fluid monitoring device (FIG. 1, 100) of the present invention depends upon the type of polymer material used to fabricate the capsule 103 component parts. The methods for injection molding, additive manufacturing, seal formation, and seam welding of polymers are well understood in the medical device industry.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Further, it is to be understood that the invention may be utilized and practiced other than as specifically described. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1-8. (canceled)

9. A method of manufacturing a fluid monitor, the method comprising:
fabricating a capsule having a first male orifice and a second male orifice, said capsule comprising a transparent enclosure or an enclosure configured with a transparent port enabling visual inspection of a fluid sampling reservoir positioned within said capsule;
incorporating into said fluid sampling reservoir any of a baffle, a flow diverter, and a filtration medium in combination with any of fluid absorbents and microfluidic channels, where fluid to be sampled for lipase is temporarily retained in said sampling reservoir;
placing in said fluid sampling reservoir a chromogenic nanofiber membrane that includes a dye chromatically responsive to the presence of at least lipase in said fluid when said fluid is temporarily retained for a period of time sufficient for said dye in said nanofiber membrane to respond to said at least lipase presence in said fluid,
positioning within said fluid sampling reservoir said chromogenic nanofiber membrane in direct and full surface contact with any of said fluid absorbents and microfluidic channels and oriented for exposure to a fluid when said fluid is present within said capsule,
wherein a visual color-change indication of said nanofiber membrane is observable absent use of wires, batteries, or external devices,
wherein, any of said fluid absorbents and microfluidic channels expose said chromogenic nanofiber membrane to fluid passing through the capsule, and
wherein, said first male orifice and said second male orifice of said capsule are each shaped to connect by insertion into a conduit usable in any of indwelling urinary catheters, chest tubes, wound drains, endotracheal tubes, central lines, hemodialysis ports, surgical site drains, and bypass grafts.

10. The method of claim 9, wherein said first male orifice and said second male orifice are shaped for removable insertion into a conduit of a negative pressure device with incorporated pump for extracting fluid from a wound or surgical site, a distal end of an intermittent urinary catheter, or an inline connection between upstream and downstream conduits.

11. The method of claim 9, wherein said selected nanofiber membrane is chromogenically reactive to at least one pathogen produced enzyme and has a structure comprising at least three adjacent layers of nanofibers, each layer comprising a plurality of aligned nanofibers oriented at oblique angles relative to nanofibers in adjacent layers, said plurality of aligned nanofibers in each layer crossing a plurality of aligned nanofibers in adjacent layers at a plurality of distinct points of intersection, forming a crossing at each distinct point of intersection consisting of three crossing nanofibers extending directionally as six radials from each distinct point of intersection in the range of 50 to 70 degrees.

12. The method of claim 9, wherein said capsule includes fluid absorbents comprising natural or synthetic material infused with a neutralizing agent to alter the pH of the fluid to be sampled, said agent selected from any of acetic acid, citric acid, and hypochlorous acid.

13. The method of claim 9, wherein said selected nanofiber membrane comprises any one or combination of poly (lactic-co-glycolic acid) (PLGA), polyvinylpyrrolidone (PVP), poly(ethyleneoxide) (PEO), polyurethane (PU), PVP/cyclodextrin, polyvinyl alcohol (PVA), polycaprolactone (PCL), cellulose, PVP/ethyl cellulose, PVP/zein, cellulose acetate, and hydroxypropyl methylcellulose (HPMC), and said capsule comprises any of Polycarbonate, Polysulfone, and Polyethylene terephthalate (PET).

20. (canceled)

23. (canceled)
